(19) United States
(12) Patent Application Publication
(10) Pub. No.: US 2013/0218007 A1
(43) Pub. Date: Aug. 22, 2013

(54) TARGETING LANDMARKS OF ORTHOPAEDIC DEVICES

(71) Applicants: Timothy J. Petteys, Bartlett, TN (US); Charles R. Baker, Lakeland, TN (US); Charles C. Heotis, Germantown, TN (US)

(72) Inventors: Timothy J. Petteys, Bartlett, TN (US); Charles R. Baker, Lakeland, TN (US); Charles C. Heotis, Germantown, TN (US)

(73) Assignee: SMITH & NEPHEW, INC., Memphis, TN (US)

(21) Appl. No.: 13/882,528

(22) PCT Filed: Oct. 31, 2011
(86) PCT No.: PCT/US11/58568

§ 371 Date: Apr. 30, 2013

Related U.S. Application Data
(60) Provisional application No. 61/408,884, filed on Nov. 1, 2010.
(60) Provisional application No. 61/546,052, filed on Oct. 11, 2011.

(51) Int. Cl. A61B 5/06; A61B 17/68

(52) U.S. Cl. CPC A61B 5/064; A61B 17/68
USPC 600/424

(57) ABSTRACT

A device for targeting a landmark of an orthopaedic implant including a housing configured to engage a mating structure for attachment of the housing to the orthopaedic implant, and an electromagnetic sensor located at a known position within the housing, wherein, when the housing is engaged with the mating structure, the position of the sensor relative to a landmark of the orthopaedic implant is known for at least five degrees of freedom.

### CROSS-REFERENCE TO RELATED APPLICATION

This application claims priority to and the full benefit of U.S. Provisional Application Ser. No. 61/408,884, filed Nov. 1, 2010, and titled “Targeting Landmarks of Orthopaedic Devices,” and U.S. Provisional Application Ser. No. 61/546,052, filed Oct. 11, 2011, and titled “Targeting Landmarks of Orthopaedic Devices,” the entire contents of which are incorporated herein by reference.

### TECHNICAL FIELD

This disclosure relates to targeting landmarks of orthopaedic devices.

### BACKGROUND

Orthopaedic device are used in the treatment of many injuries or conditions. For example, treatment of certain bone fractures involves stabilizing selected portions and/or fragments of bone using an implantable orthopaedic plate and/or an implantable orthopaedic nail, and bone screws or pins. As another example, joints can be fused or otherwise immobilized using plates and/or nails secured with bone screws or pins.

In some instances, it is necessary or beneficial to target a hidden landmark of an orthopaedic implant. For example, some procedures involve placement of bone screws or pins through selected apertures of an implanted orthopaedic device. Such targeting can be accomplished in some cases using radiographic imaging. Unfortunately, radiographic imaging can be undesirable for various reasons. For example, exposure to radiation energy used in the imaging process can be harmful to a patient as well as to those treating the patient or assisting those treating the patient. Additionally, radiographic imaging can be expensive and time-consuming, as well as potentially inaccurate, or less accurate than desired.

Recently, electromagnetic-based targeting of orthopaedic implants has been employed to determine a relative location and orientation of a tool and a feature of an implanted orthopaedic device. For example, distal locking holes of an implanted intramedullary nail can be targeted for drilling and fixation using a locking screw with an electromagnetic targeting system, such as the TRIGEN® SURESHOT® distal targeting system offered by SMITH & NEPHEW®.

### SUMMARY

In one general aspect, a device can include a position sensor and a housing. The housing can be configured to engage a mating structure that is coupled to or formed on an orthopaedic implant. When the housing is engaged to the mating structure, the position sensor can be used to determine the position of a landmark of the orthopaedic implant.

In another general aspect, a device for targeting a landmark of an orthopaedic implant includes a housing configured to engage a mating structure for attachment of the housing to the orthopaedic implant. The device also includes an electromagnetic sensor located at a known position within the housing, wherein, when the housing is engaged with the mating structure, the position of the sensor relative to a landmark of the orthopaedic implant is known for at least five degrees of freedom.

Implementations may include one or more of the following features. For example, the housing includes one of a generally cylindrical outer surface having a detent and a ball plunger to fixedly attach the housing to the implant. The housing includes a split end that is expandable to engage the mating structure and fixedly attach the housing to the implant. The housing includes an outer surface having a compressible member configured to engage a groove to fixedly attach the housing to the implant. The housing includes a tapered outer surface configured to engage a seat to fixedly attach the housing to the implant. The housing defines a central longitudinal axis, the housing having a curvature along the central longitudinal axis of the housing. The mating structure includes a polygonal external portion and wherein the housing includes a complimentary polygonal portion for mating with the polygonal external portion.

In another general aspect, a method of targeting a landmark of an orthopaedic device includes locating a first landmark of the orthopaedic device using a landmark identifier and a first electromagnetic field sensor. The landmark identifier has an electromagnetic field generator, and the first electromagnetic field sensor being within a working volume of the electromagnetic field generator when locating the first landmark The method includes placing a second electromagnetic field sensor in the working volume, and locating a second landmark of the orthopaedic device using the landmark identifier and the second electromagnetic field sensor.

Implementations may include one or more of the following features. For example, the first electromagnetic field sensor is located outside the working volume of the electromagnetic field generator when locating the second landmark The first landmark is a hole. The orthopaedic device is a bone plate Attaching the second electromagnetic field sensor includes accessing the hole and attaching the second electromagnetic field sensor to the orthopaedic device via the hole. The hole is accessed when the orthopaedic device is implanted in a patient. The hole is a threaded hole, and wherein attaching the second electromagnetic field sensor includes engaging a drill sleeve with the threaded hole and attaching the second electromagnetic field sensor to the drill sleeve. The method further includes disposing the first electromagnetic field sensor in a known position relative to the first landmark

The method further includes calibrating the first electromagnetic field sensor. Calibrating the first electromagnetic field sensor includes the use of at least one of the second electromagnetic field sensor and the electromagnetic field generator. The method further includes providing a housing for mounting the first electromagnetic field sensor and the housing includes at least two mating structures configured for engaging with pre-selected sites on the implant. The method further includes providing a housing for mounting the first electromagnetic field sensor wherein the housing includes a feature which mates with a pre-selecting feature in the implant. The method further includes calibrating the second electromagnetic field sensor. Calibrating the second electromagnetic field sensor includes calibrating a signal received from the second electromagnetic field sensor based on a signal received from the first electromagnetic field sensor when the first and second electromagnetic field sensors are located within the operating volume of the electromagnetic field generator. The method further includes changing a global reference frame of a targeting system from a position of the first electromagnetic field sensor to the position of the second electromagnetic field sensor.

In another general aspect, a method of targeting a landmark of an orthopaedic implant includes fixedly attaching an electromagnetic field sensor in a first location, the position of the electromagnetic field sensor relative to a first landmark of the orthopaedic implant being known for multiple degrees of freedom. The method includes determining a position of the electromagnetic field sensor for an unknown degree of freedom and calibrating the electromagnetic field sensor using at least one of an electromagnetic field generator and an electromagnetic sensor. The method also includes targeting a second landmark of the orthopaedic implant using the calibrated electromagnetic field sensor and the electromagnetic field generator.

Implementations may include one or more of the following features. For example, the method further includes implanting the orthopaedic implant in a patient after calibrating the electromagnetic field sensor. The method includes implanting the orthopaedic implant in a patient before calibrating the electromagnetic field sensor. The method further includes fixedly attaching the electromagnetic field generator to the orthopaedic implant in a known position relative to a third landmark of the orthopaedic implant, wherein the electromagnetic field sensor is calibrated when the electromagnetic field generator is attached to the orthopaedic implant. The second landmark is the same as the third landmark. The method further includes fixedly attaching a second electromagnetic field sensor to the orthopaedic implant in a known position relative to a third landmark of the orthopaedic implant, wherein the electromagnetic field sensor is calibrated when the second electromagnetic field sensor is attached to the orthopaedic implant. The second landmark is the same as the third landmark. The method further includes fixedly attaching the electromagnetic field sensor to the orthopaedic implant includes attaching a drill sleeve to a hole of the orthopaedic implant and attaching a housing to the drill sleeve, the electromagnetic field sensor being attached to the housing. The position of the electromagnetic field sensor relative to the hole of the orthopaedic implant is known for three translational degrees of freedom and for two rotational degrees of freedom. Calibrating the electromagnetic field sensor comprises determining a rotational position of the electromagnetic field sensor in a third rotational degree of freedom. Fixedly attaching the electromagnetic field sensor to the orthopaedic implant includes attaching an insertion handle to the orthopaedic implant.

In another general aspect, a method of confirming acceptable positioning of a tool relative to an orthopaedic stabilization structure includes receiving a signal from a sensor, the signal being indicative of a position of the tool relative to a landmark of the orthopaedic stabilization structure. The method includes determining the position of the tool relative to the landmark and comparing the position of the tool to an acceptable range of positions of a fastener relative to the landmark. The method includes determining that the position of the tool relative to the landmark corresponds to an acceptable position within the range of positions of the fastener relative to the landmark, and outputting on a graphical user interface an indication that the position of the tool relative to the landmark is acceptable.

Implementations may include one or more of the following features. For example, outputting includes outputting one or more elements selected from the group consisting of: elements representing the angle of a drill bit relative to the central through axis of the variable-angle hole, one or more elements representing acceptable positions of the tool relative to the landmark, one or more elements representing unacceptable positions of the tool relative to the landmark, a numeric representation of the angle of a drill bit relative to the central through axis of the variable-angle hole, a numeric representation of the maximum acceptable insertion angle of the fastener, an element indicating that the current position of the tool is acceptable, and an element indicating that the current position of the tool is unacceptable. The orthopaedic stabilization structure includes an orthopaedic bone plate, the landmark is a variable-angle hole of the orthopaedic bone plate, and the fastener is a bone screw configured for variable-angle insertion in the variable-angle hole. The landmark is a variable-angle locking hole. The tool includes a drill bit, and wherein comparing includes comparing an angle of the drill bit relative to a central through axis of the variable-angle hole to an acceptable insertion angle of the fastener in the variable-angle hole. Outputting includes outputting one or more elements selected from the group consisting of: elements representing the angle of the drill bit relative to the central through axis of the variable-angle hole, a graphical representation of an acceptable conical range of a variable angle or variable angle locking screw, one or more elements representing acceptable positions of the tool relative to the landmark, one or more elements representing unacceptable positions of the tool relative to the landmark, a numeric representation of the angle of the drill bit relative to the central through axis of the variable-angle hole, a numeric representation of the maximum acceptable insertion angle of the fastener, an element indicating that the current position of the tool is acceptable, and an element indicating that the current position of the tool is unacceptable.

In another general aspect, a landmark identifier for use in targeting a landmark of an orthopaedic implant includes a housing and at least one electromagnetic field generator disposed within the housing. The electromagnetic field generator is operable to generate a working volume. The landmark identifier is programmed for operation in at least a first mode and a second mode, wherein the working volume when operating in the first mode differs from the working volume when operating in the second mode.

In another general aspect, method of providing tracking information includes tracking a position of an instrument relative to an orthopaedic implant, determining the position of a trajectory defined by the implant relative to the orthopaedic implant, and indicating, on a user interface, the position of the trajectory defined by the instrument relative to the orthopaedic implant.

Implementations may include one or more of the following features. For example, the method includes identifying one or more transfixion element that are indicated for use with the orthopaedic implant along the trajectory, and providing information that identifies the one or more transfixion elements that are indicated for use. The method includes identifying one or more component types that are indicated for use with the orthopaedic implant at the trajectory defined by the instrument, and providing information that identifies the one or more component types that are indicated for use. The method includes determining a depth that an instrument has drilled relative to the orthopaedic implant, and indicating the depth on the user interface. Determining a depth that an instrument has drilled relative to the orthopaedic implant includes determining the relative position of a drill relative to a drill guide. Determining the relative position of the drill and the drill guide includes determining a relative position of a first fiducial coupled to the drill and a second fiducial coupled to the drill guide. The method includes tracking the position and depth that an instrument drills relative to the orthopaedic implant, and storing information indicating the position and depth that are drilled by the instrument. The method includes storing data that indicates a position of a drilled hole or an inserted screw, determining that a trajectory of the instrument interferes with the drilled hole or the inserted screw, and in response to determining that the trajectory of the instrument interferes with the drilled hole or the inserted screw, providing an indication of the interference. The method includes determining a maximum length that a transfixion element can extend along the trajectory without interfering with the drilled hole or the inserted screw, and providing an indication of the maximum length. The method includes determining that a fiducial coupled to an instrument is not being accurately tracked by a tracking system, and indicating on the user interface that the instrument is not being accurately tracked by the tracking system. The method includes providing one or more configuration indicators that identify instruments or implants that are being tracked by a tracking system. Tracking a position of an instrument relative to an orthopaedic implant includes tracking a relative position of a first optical reference coupled to the instrument and a second optical reference coupled to the orthopaedic implant. Tracking a position of an instrument relative to an orthopaedic implant includes tracking a relative position of a first electromagnetic field sensor coupled to the instrument and a second electromagnetic field sensor coupled to the orthopaedic implant.

The details of one or more implementations are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a perspective view of a landmark identifier.

FIG. 2 is a perspective view of a sensor assembly and a drill sleeve.

FIGS. 3-6 are perspective views of a system for targeting landmarks.

FIG. 7 is a perspective view of a system for targeting landmarks.

FIGS. 8A-11A are side views of housings of sensor assemblies.

FIG. 11B is a cutaway view of a drill sleeve.

FIG. 12A is a cutaway view of a drill sleeve and an attachment member.

FIG. 12B is a top view of a clip of the attachment member of FIG. 12A.

FIGS. 13 and 14 are perspective views of housings of sensor assemblies.

FIG. 15 is a perspective view of a system for targeting landmarks.

FIG. 16A is a perspective view of a guide of the system of FIG. 15.

FIG. 16B is a perspective view of an attachment of the system of FIG. 15.

FIG. 16C is a perspective view of a drill of the system of FIG. 15.

FIGS. 17 and 18 are examples of user interfaces of a control unit of the system of FIG. 15.

### DETAILED DESCRIPTION

A system for targeting landmarks of orthopaedic implants or other orthopaedic devices includes a landmark identifier that is configured for attachment to an orthopaedic tool and that includes an electromagnetic field generator that is operable to generate an electromagnetic field having known properties. The system also includes one or more electromagnetic sensors and/or field generator(s) configured for attachment to an orthopaedic implant or other orthopaedic device to be targeted and the system includes a control unit configured to drive the electromagnetic field generator, receive output signals from the sensor(s), and display relative positions of the orthopaedic device and the landmark identifier. For example, the landmark identifier, sensors, and control unit can include features as described in WIPO International Publication Nos. WO2008/106593 and WO2009/108214, and as described in U.S. patent application Ser. Nos. 12/758,747 and 12/768,689, each of which is incorporated herein in its entirety.

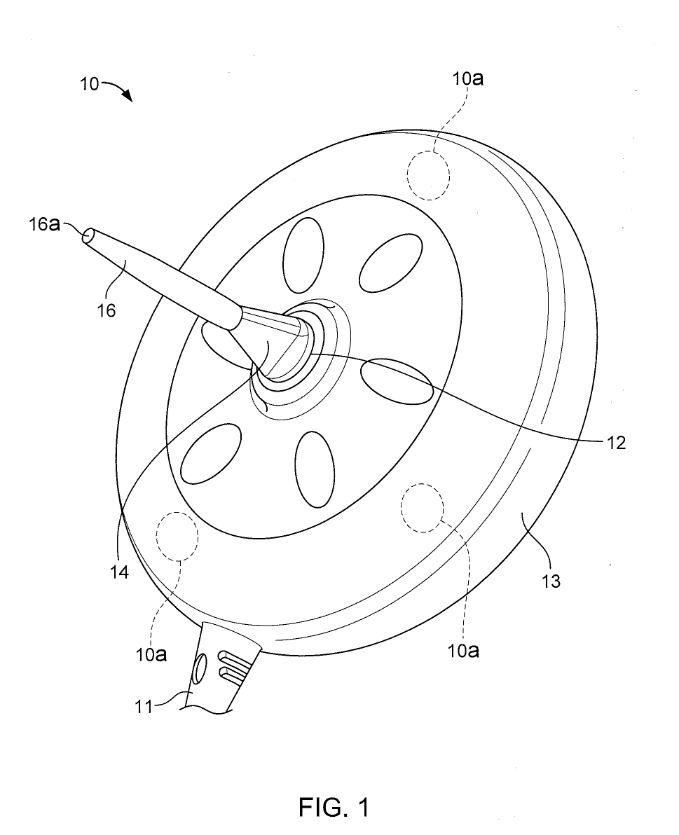

Now referring to FIGS. 1-3, a landmark identifier 10 includes an electromagnetic field generator 10a that produces an electromagnetic field that has known characteristics. The electromagnetic field generator 10a is located within a housing 13 of the landmark identifier 10. The electromagnetic field generator 10a includes one or more coils or other components that produce electromagnetic fields. The generated electromagnetic fields can be detected by one or more electromagnetic sensors, and, based on the output of the sensors, the position (including the location and the orientation) of the sensors relative to the landmark identifier 10 can be determined.

The useful range of the landmark identifier 10 is a three-dimensional region around the landmark identifier 10, referred to as the working volume of the landmark identifier 10. The size and shape of the working volume is based on the characteristics of the electromagnetic fields produced by the electromagnetic field generator 10a and can be modified to be larger or smaller based on the need for targeting accuracy. For example, when targeting a hole in an intramedullary nail, it may be desirable to have high degree of accuracy due to the fact that the hole is hidden inside a bone. In some implementations, the working volume is smaller as a result of increasing the degree of accuracy. For targeting a hole in a bone plate, it may not be necessary to have very high degree of accuracy due to the location of the hole of the bone plate outside a bone, where it can be exposed for visual confirmation of its location. As a result, the working volume can be made much larger than in some intramedullary nail targeting applications. The larger working volume makes it possible to target a larger number of holes in the working volume. In some implementations, the working volume is a volume that surrounds the landmark identifier 10. For example, the landmark identifier 10 can be generally centrally located within the working volume, and the working volume for some implementations, such as targeting holes of a bone plate, can extend approximately 50 cm or more in width and approximately 40 cm or more in depth and located at a distance of about 5 cm from the landmark identifier 10. A drill sleeve, for example, will have a length of more than 5 cm to ensure that it is positioned within the working volume.

An electromagnetic field sensor assembly 20 located within the working volume of the landmark identifier 10 is able to generate output signals that indicate strength or intensity of the electromagnetic field generated by the landmark identifier 10. The output signals can be used to accurately determine a location and orientation of the landmark identifier 10 relative to the sensor. A sensor located outside the working volume of the landmark identifier 10, on the other hand, generally does not or may not receive adequate electromagnetic energy from the landmark identifier 10 to generate output signals that can be used to accurately determine the position of the landmark identifier 10. The shape and size of the working volume of the landmark identifier 10 depends in part on the configuration of the electromagnetic field generator 10a, specific characteristic of the operation of the electromagnetic field generator 10a, such as characteristics of a driving signal, and other factors.

In some implementations, the landmark identifier 10 is programmed to operate in multiple modes and is controlled by a chip mounted on an electronic circuit board inside the landmark identifier 10. For example, in a first mode of operation, the identifier 10 produces a first working volume. In a second mode of operation, the identifier 10 produces a working volume having different characteristics than the working volume produced in the first mode. For example, the working volume produced in the second mode can be larger or smaller than the working volume produced in the first mode. Additional modes and variations between modes are possible.

The landmark identifier 10 can include a wired or wireless link to a control unit 40 (FIG. 3) to receive power and control signals to control the operation of the electromagnetic field generator 10a. For example, the landmark identifier 10 can include a cable 11 that provides a connection to the control unit 40.

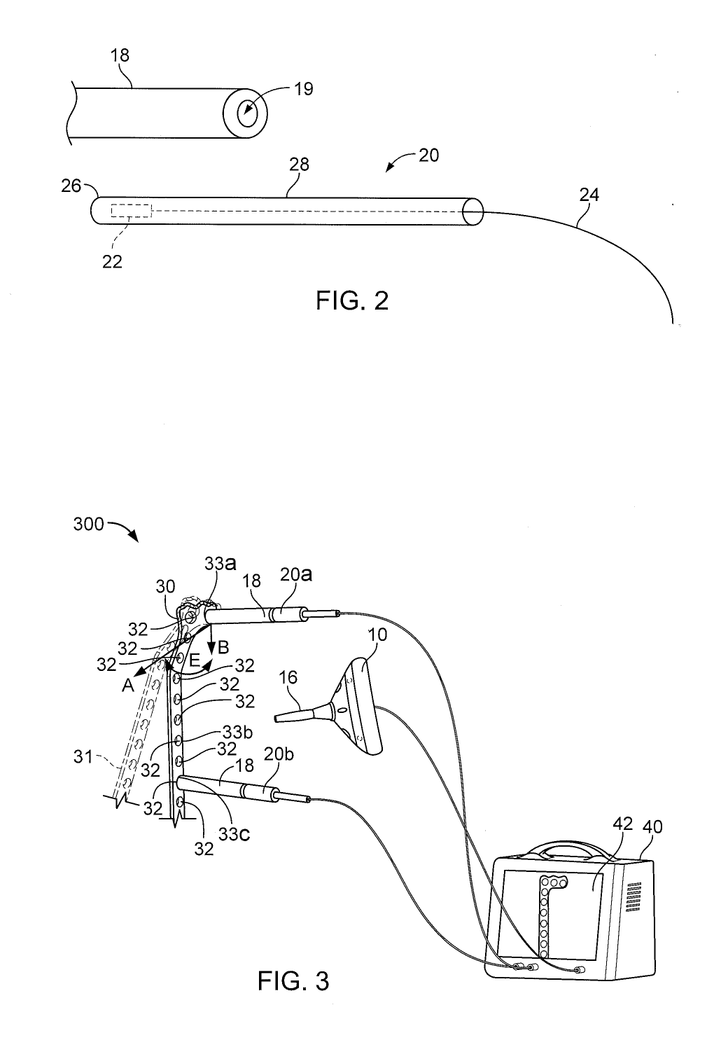

An operator, such as a surgeon, can grip the landmark identifier 10 by the housing 13 to position the landmark identifier 10 relative to a patient, an orthopaedic device, and/or a sensor, such as the electromagnetic sensor assembly 20 (FIG. 2). The landmark identifier 10 can also include a coupling member 12 to which tools and other attachments may be coupled. Using the coupling member 12, tools and other devices can be attached or guided by the landmark identifier 10. For example, the coupling member 12 can receive a drill guide attachment 14 coupled to a drill guide 16. The landmark identifier 10 can be used to position the drill guide 16 so that a drill bit inserted through the drill guide 16 is guided to the position required by or appropriate for a medical procedure.

As shown in FIG. 2, the electromagnetic field sensor assembly 20 includes an electromagnetic field sensor 22, a sensor lead 24, and a housing 28. The electromagnetic field sensor 22 may be, for example, an inductive sensor that is configured to respond to an electromagnetic field produced by a landmark identifier 10 by outputting one or more induced electrical currents. The sensor 22 can be capable of producing signals that allow the position of the landmark identifier 10 to be determined. For example, the sensor 22 can include two or more inductive coils that each outputs an induced electrical current. The outputs of the sensor 22 allow determination of the location and orientation of the sensor 22 in up to six degrees of freedom, such as along three translational axes, generally called X, Y, and Z, and three angular orientations, generally called pitch, yaw, and roll, which are defined as rotation about the three translational axes.

The sensor 22 includes a connection to transmit the output signals, or data related to the signals. For example, a sensor lead 24 provides a wired connection for transmission of an output of the sensor assembly 20. The sensor lead 24 can carry signals produced by the sensor 22 in response to electromagnetic fields. In some implementations, the connection can include a wireless transmitter. Additionally, the sensor lead 24 can include more than one connection, and the sensor lead 24 can carry power and control signals in addition to signals or data, and bi-directional communication is possible. For example, information regarding calibration of the sensor 22 can be stored in a storage device of the sensor assembly 20.

The sensor 22 is secured to the housing 28 to maintain the position of the sensor 22 relative to the housing 28. For example, the sensor 22 can be fixedly attached to a housing 28 at a known position of the housing 28. Maintaining the position of the sensor 22 relative to the housing 28 allows the position of the sensor 22 relative to a landmark of an orthopaedic device to be known for at least five degrees of freedom when the housing 28 is attached to the orthopaedic device directly or indirectly. The sensor 22 is coupled to the housing 28 at a location of the housing 28 that is likely to be positioned near a landmark, for example, a distal end 26 of the housing 28, such that the sensor 22 is likely to be located within the working volume of the landmark identifier 10 when attempting to target the landmark.

The housing 28 of the sensor assembly 20 is configured to engage a mating structure of an orthopaedic device or a structure connected to the orthopaedic device, such as a drill sleeve 18 to secure the sensor assembly 20 to the orthopaedic device, for example, to an orthopaedic implant 30 (FIG. 3). For example, the housing 28 may be coupled to a mating structure to prevent the housing 28 from disengaging from the orthopaedic implant 30 or altering the orientation of the sensor 22 while the sensor 22 is in use.

The drill sleeve 18 can be configured to receive the sensor housing 28 such that when the drill sleeve 18 is attached to the orthopaedic implant 30, the sensor housing 28 and the sensor 22 are fixedly attached to the orthopaedic implant 30. The sensor assembly 20 can attach to the drill sleeve 18 by, for example, entering a through hole 19 of the drill sleeve 18. The drill sleeve 18 can attach to the orthopaedic implant 30 by engaging a threaded end (not shown) of the drill sleeve 18 with a threaded aperture 32 of the orthopaedic implant. In some implementations, such as where the threaded aperture 32 or a non-threaded aperture is not clocked, meaning that there are no features in or around the aperture to repeatedly and consistently fix the location and orientation of the sensor 22 relative to a reference point on the orthopaedic implant. For example, attaching the sensor 22 to the orthopaedic implant 30 may secure the sensor 22 in a position relative to the orthopaedic implant 30 that is known for five degrees of freedom. In particular, the rotational position of the sensor 22 may be unknown due to a threaded engagement of the drill sleeve 18 with the orthopaedic implant 30 and possibly due to rotation of the housing 28 within the through hole 19. When the position of the sensor 22 is not known for one or more degree of freedom, the sensor 22 can be calibrated before use in targeting one or more landmarks of the orthopaedic device 30.

As illustrated in FIG. 3, a targeting system 300 includes a control unit 40, a first sensor assembly 20a, a second sensor assembly 20b (or a second field generator assembly for use in calibrating the first sensor assembly 20a), and the landmark identifier 10. The system 300 can be used for targeting landmarks of an orthopaedic device, such as an orthopaedic implant or an orthopaedic stabilization structure 30. The orthopaedic implant 30 illustrated in FIG. 3 is a bone plate and can be attached to a fractured bone to provide alignment and support for bone portions during a healing process. As other examples, orthopaedic devices that can be targeted using the system 300 include intramedullary nails, bone plates, prosthetic joint components, and external fixation devices.

The orthopaedic implant 30 includes multiple landmarks. As examples, a landmark may be a structure, a void, a boss, a channel, a detent, a flange, a groove, a member, a partition, a step, an aperture, a bore, a cavity, a dimple, a duct, a gap, a notch, an orifice, a passage, a slit, a hole, or a slot. For example, the orthopaedic implant 30 includes various holes 32 as landmarks. The holes 32 may be, for example, variable-angle holes, variable-angle locking holes, or fixed-angle locking holes.

During the implantation process and afterward, the precise location and orientation of landmarks may be needed to correctly position a drill bit, nail, screw, or other device. However, landmarks may be covered by tissue and may be difficult to locate. Additionally, jigs or other means for determining an angle of a tool relative to a landmark may be difficult and time consuming to use, and may not provide a desired degree of accuracy. The landmark identifier 10 can be used to target landmarks, or in other words, to determine the position of the landmarks, even when the landmarks are covered by tissue. The landmark identifier 10 can also be used to determine a position, of a tool, such as a drill or a fastener, relative to a landmark even when the landmark is exposed.

The control unit 40 of the system 300 controls the operation of the landmark identifier 10 and receives inputs from one or more of the sensor assemblies 20a, 20b. The control unit 40 also includes a user interface 42 that provides information to an operator of the system 300. The control unit 40 includes a processor that is configured to determine the location and orientation of the sensor 22 relative to landmarks of the orthopaedic implant 30 based on the input from the sensor assemblies 20a, 20b and information regarding the signal that controls the electromagnetic field generator 10a. The determination is made based on a known positional relationship between the sensor assemblies 20a, 20b and the landmarks and a determined position of the landmark identifier 10 relative to the sensor assemblies 20a, 20b.

In some implementations, only one sensor assembly, such as the sensor assembly 20a, is required for targeting some landmarks, such as those within limited distance from the sensor assembly 20a when it is attached to the orthopaedic implant 30. As mentioned above, it may be necessary to calibrate the sensor assembly 20a before targeting landmarks, such as where the attachment of the sensor assembly 20a to the orthopaedic implant 30 results in an unknown position of the sensor 22 relative to the orthopaedic implant 30 in at least one degree of freedom. In some implementations, the first sensor assembly 20a of the landmark identification system 300 can be calibrated before the orthopaedic implant 30 is implanted. For example, an unknown orientation of the sensor 22 relative to the implant 30 may be determined through calibration of the first sensor assembly 20a by use of a second sensor assembly or a field generator before an orthopaedic device is implanted. The calibration as described below can also be done during the implantation process.

To calibrate a first sensor assembly 20a for the unknown sixth degree of freedom, the first sensor assembly 20a is fixedly attached to the orthopaedic implant 30. The first sensor assembly 20a is attached so that the position of the electromagnetic sensor 22 relative to a first landmark 33a of the bone plate 30 is known for multiple degrees of freedom. As shown, the position of the sensor 22 is known for three orthogonal translational degrees of freedom when the housing 28 is inserted in the drill sleeve 18 and the drill sleeve 18 is threaded completely into the first landmark 33a. Alternatively, the sensor assembly 20a may be attached directly to the first landmark 33a. The position of the sensor 22 of the first sensor assembly 20a is then determined for an unknown degree of freedom. For example, the second sensor assembly 20b, or a field generator as described below, is fixedly attached to the bone plate 30 at a location that is known relative to a third landmark 33c of the bone plate 30. The first sensor assembly 20a and the second sensor assembly 20b can be located so that both sensor assemblies 20a, 20b are simultaneously within the working volume of the landmark identifier 10 during calibration. Thus, when the landmark identifier 10 is controlled to generate an electromagnetic field, each of the sensor assemblies 20a and 20b outputs signals or data to the control unit 40 to allow the control unit 40 to determine the location and orientation of the sensor 22 of the first sensor assembly 20a relative to the landmark 33a.

For example, in determining the position of the sensor 22 relative to the landmark 33a, the control unit 40 can access information about the shape of the orthopaedic implant 30 and the locations of the features of the orthopaedic implant 30. Additionally, the control unit 40 can access information regarding the locations of the sensors 22 of the first and second sensor assemblies 20a and 20b. For example, the first and second sensor assemblies 20a and 20b can be attached to pre-selected landmarks 33a and 33c, or information regarding the landmarks 33a and 33c to which the sensor assemblies 20a and 20b are attached can be input to the control unit 40, such as by a user touching a portion of the interface 42 to indicate a landmark to which the sensor assemblies are attached. The signals of the first sensor assembly 20a and the second sensor assembly 20b can be used in combination with the information regarding known positions of the first sensor assembly 20a and the second sensor assembly 20b to determine the location and orientation of the first sensor assembly 20a for one or more unknown degrees of freedom, such as a rotational degree of freedom that was not previously known.

Calibrating the first sensor assembly 20a can include storing calibration data in the control unit 40 and/or can include storing calibration data in a first sensor assembly 20a. For example, before calibration, the control unit 40 may interpret a signal from the first sensor assembly 20a to indicate an orientation of a bone plate 30 along arrow A. Even though the location and orientation of the first sensor assembly 20a may be interpreted accurately, without calibration, the signal from the first sensor assembly 20a may be inaccurately interpreted by the control unit 40, for example, to inaccurately indicate that the bone plate 30 is oriented at an inaccurate orientation 31. Nevertheless, using the position signal of the second sensor assembly 20b, the control unit 40 can determine that the signal from the first sensor assembly 20a refers not to arrow A, but rather to arrow B. Using this information, the control unit 40 can determine an offset, such as angle E, to calibrate the signal received from the first sensor assembly 20a. After calibration, the control unit 40 is able to determine the correct location and orientation of the bone plate 30 based on the calibration data and input from the first sensor assembly 20a, without further input from the second sensor assembly 20b.

After calibrating the first sensor assembly 20a, the second sensor assembly 20b can be removed from the orthopaedic implant 30 and the orthopaedic implant 30 can be implanted in a patient. After implantation, as will be described in greater detail below, the calibrated sensor assembly 20a and the landmark identifier 10 can be used to target a landmark 32 and others of the orthopaedic implant 30 that are located in the working volume. Landmarks which are outside the working volume can be targeted via a second sensor, referred to as a leapfrog method, as described below.

Additionally, or alternatively, the first sensor assembly 20a can be calibrated without use of the second sensor assembly 20b by fixedly attaching the landmark identifier 10 to the orthopaedic implant 30. The landmark identifier 10 can be fixedly attached at a known position relative to a landmark of the bone plate 30, such as by attaching a calibration member to the coupling member 12 and attaching the calibration member to a selected or pre-selected landmark, such as the landmark 33b. The known location of the landmark identifier 10 can be used as a reference point to calibrate the first sensor assembly 20a in a manner similar to that described above with respect to the known position of the second sensor assembly 20b.

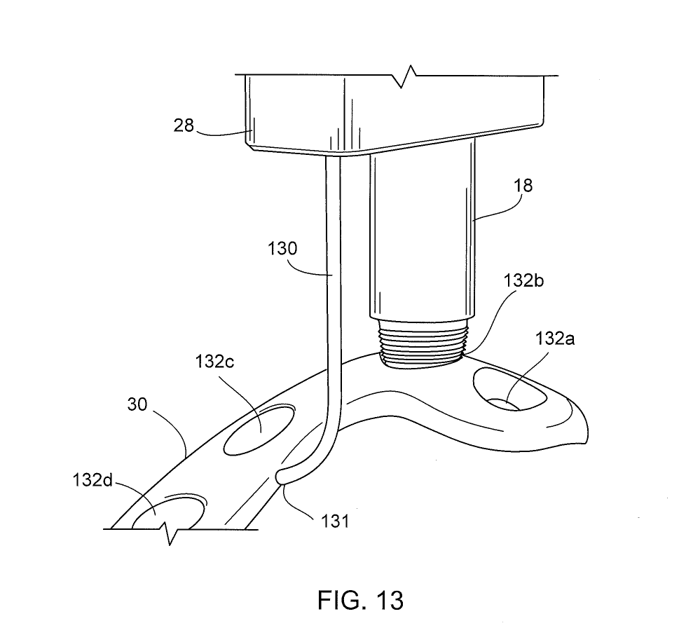

Alternatively, the first sensor assembly 20a can be calibrated without use of the second sensor assembly 20b or a field generator by providing the distal end of the housing 28 with at least two separate mating structures for engaging with two pre-selected landmarks or reference structures in or on the orthopaedic implant. For example, as shown in FIG. 13, the housing 28 can be engaged with the orthopaedic implant 30 such that the the position of the housing with respect to the orthopaedic implant is known for six degrees of freedom, making calibration with a second sensor assembly 20b or a field generator unnecessary. The housing 28 can be engaged with an orthopaedic implant 30 by external attachment to a drill sleeve 18. The orthopaedic implant 30 can include a number of landmarks 132a-132d, and the drill sleeve 18 can be coupled to the orthopaedic implant 30 at a particular known landmark 132b.

The housing 28 can include an extension 130 that is configured to contact the orthopaedic implant 30 at a particular position of the housing 28 with respect to the orthopaedic implant 30. For example, the extension 130 may include an end 131 that contacts the side of the orthopaedic implant 30. Contact of the end 131 with the orthopaedic implant 30 indicates that the housing 28, and thus a sensor 22 coupled to the housing 28, is disposed in a known position relative to the orthopaedic implant 30.

In some implementations, the drill sleeve 18 coupled to the housing 28 engages the orthopaedic implant 30 with a threaded connection. The extension 130 can be configured to permit the drill sleeve 18 engage the landmark 132b by rotating with respect to the orthopaedic implant 30. At a particular known position, the extension 130 contacts the orthopaedic implant 30 to impede further rotation of the drill sleeve 18 and the housing 28, and the rotational position of the housing 28 is known with respect to the orthopaedic implant 30. Because the sensor 22 coupled to the housing 28 provides position information for five degrees of freedom and the known rotational position indicates the sixth degree of freedom, calibration of the sensor 22 with a second sensor assembly or a field generator is not necessary.

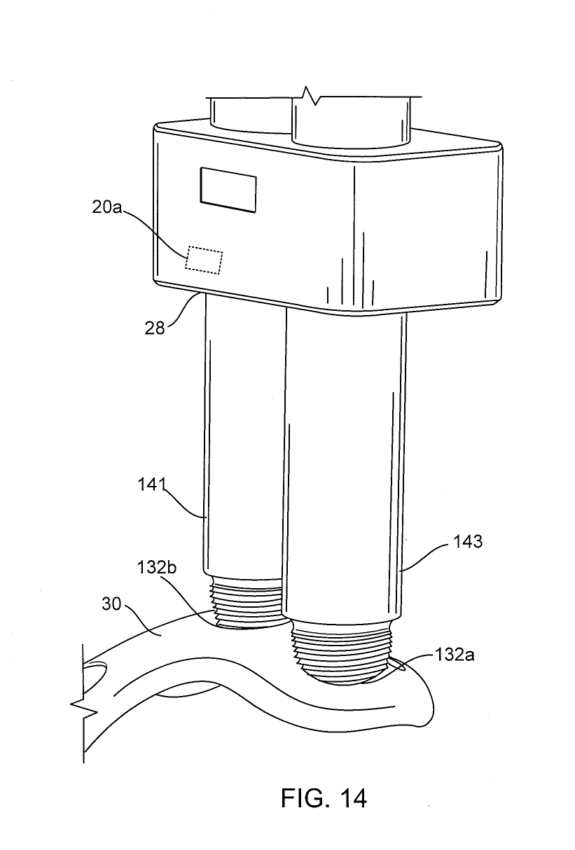

Similarly, as shown in FIG. 14, the housing 28 can include two portions 141 and 143 configured to engage two separate holes 132a and 132b, or other structures of the orthopaedic implant 30. The two separate mating structures 141 and 143 of the housing 28 can be configured such that the mating structures can only simultaneously engage the holes 132a and 132b in a single position with respect to the orthopaedic implant 30. Thus, because the housing 28 is known to be in the single position when both mating structures 141 and 143 engage the holes 132a and 132b of the orthopaedic implant 30, the position of the first sensor assembly 20a can be known for six degrees of freedom based on proper engagement of the housing with the orthopaedic implant without a separate calibration procedure.

In some implementations, as described in greater detail below with reference to FIG. 7, the first sensor assembly 20a can be calibrated without use of the second sensor assembly 20b or a field generator by coupling the sensor assembly 20a or the sensor 22 to a handle 60 that can be engaged with the orthopaedic implant 30 in a known position. Because the position of the sensor 22 is known with respect to the handle 60, the position of the sensor 22 is also known with respect to the bone plate 30 when the handle 60 is engaged with the orthopaedic implant 30 in the known position.

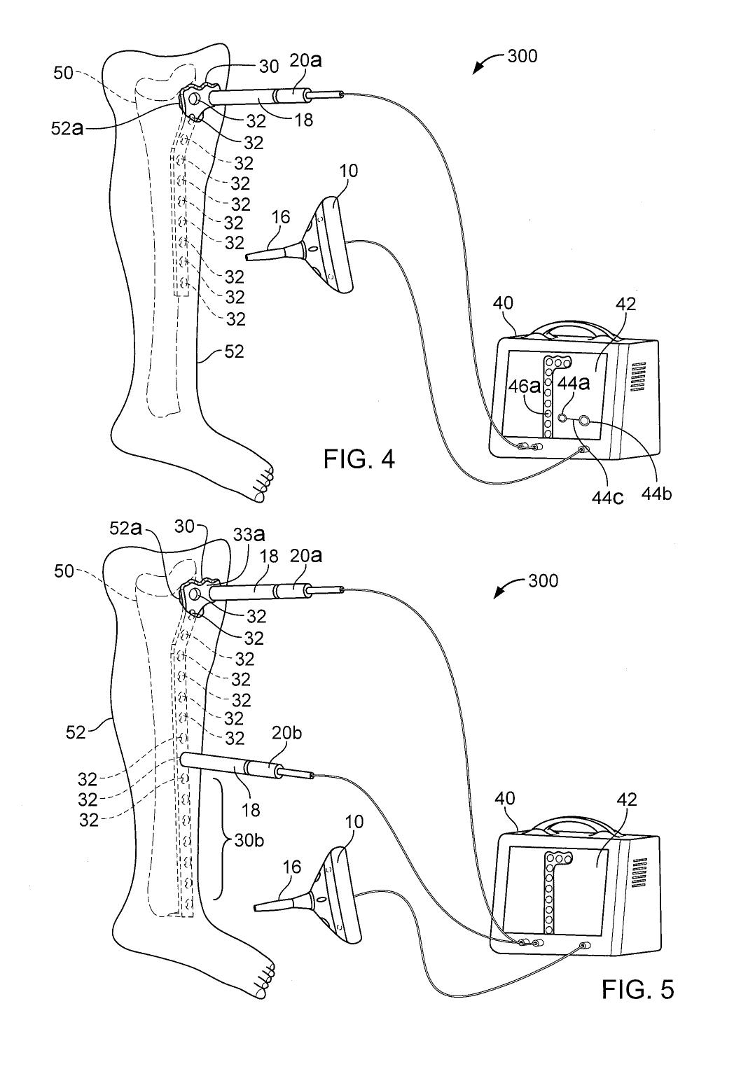

Now referring to FIG. 4, after calibration of the first sensor assembly 20a and implantation of the orthopaedic implant 30, the landmark identifier 10 and the control unit 40 can be used to target additional landmarks 32 of the orthopaedic implant 30 that are located within the working volume. For example, the orthopaedic implant 30 may be implanted next to a bone 50 of a patient beneath the patient's skin 52 by inserting the orthopaedic implant through an incision 52a in the patient's skin. Thus, the holes 32 of the bone plate 30 are not visible because they are obscured by the patient's skin 52 and other tissues. Because the position of the first sensor assembly 20a relative to the landmark identifier is known for six degrees of freedom, however, landmarks 32 of the orthopaedic implant 30 that are within the working volume when the sensor assembly 20a is also within the working volume can be located based on known positions of the landmarks 32 relative to the sensor assembly 20a.

To target one of the landmarks 32, the landmark identifier 10 is positioned near the orthopaedic implant 30, such as with a tip 16a of the drill guide 16 in contact with the patient's skin When the first sensor assembly 20a is located within the working volume of the landmark identifier 10, and the electromagnetic field generator 10a produces an electromagnetic field, the control unit 40 receives signals produced by the first sensor assembly 20a that indicate the position of the first sensor assembly 20a relative to the landmark identifier 10. Using the signals from the first sensor assembly 20a, the control unit 40 can determine the position of the landmark identifier 10 relative to landmarks 32 of the orthopaedic implant 30. The control unit 40 outputs information about the position of the landmark identifier 10 relative to landmarks 32 of the orthopaedic implant 30 on the user interface 42. Based on the user interface 42, a surgeon or other operator can place the landmark identifier 10 in a position where the interface 42 indicates that the tip 16a of the drill guide 16 is directly above a selected landmark 32 of the orthopaedic implant 30. In some implementations, the interface 42 includes a first identifier element 44a, such as a first circle, that indicates a position of the distal tip 16a of the drill guide 16. Thus, when the first identifier element 44a is in alignment with a landmark element 46a that corresponds to, and represents a targeted landmark 32, the interface 42 indicates that the tip 16a of the drill guide 16 is directly above the landmark 32 represented by the landmark element 46a. The interface 42 can also include different graphical elements, and can include audio or haptic outputs.

When the location of the landmark 32 is known, the landmark 32 can be exposed, such as by making an incision in the area of the tip 16a of the drill guide 16 when the first identifier element 44a is aligned with the landmark element 46a as indicated on the user interface 42. A provisional fixation pin, a non-locking bone screw, a locking bone screw, or a variable locking bone screw can then be engaged with the patient's bone and/or the landmark 32. Additionally, a drill or other tool can be used to create a hole in the patient's bone to receive one or more of the fasteners mentioned above.

The interface 42 of the control unit 40 can also indicate a current angular position of the landmark identifier 10 relative to the orthopaedic implant 30 or a landmark 32 to confirm acceptable positioning of a tool relative to the orthopaedic implant 30. For example, the control unit 40 can display a current angle of the drill guide 16 relative to a variable angle locking hole of the orthopaedic implant 30 so that an operator, such as a surgeon, can confirm that a hole drilled in the patient's bone 50 will result in an acceptable angle for a variable angle locking fastener. In some implementations, the interface 42 includes a second identifier element 44b, such as a second circle, that represents a proximal portion of the landmark identifier 10, and a third identifier element 44c that represents an axis from the first identifier element 44a to the second identifier element 44b. As illustrated in FIG. 4, as the first identifier element 44a and the second identifier element 44b approach one another, the angle of the landmark identifier 10 approaches zero degrees from a reference axis, such as a central through axis of a hole of the orthopaedic implant. Thus, when the first identifier element 44a and the second identifier element 44b are concentric, the landmark identifier 10 is parallel to the reference axis.

The control unit 40 receives a signal that indicates a position of the landmark identifier 10 relative to a landmark 32 of the orthopaedic implant 30. The signal can be received from the sensor 22, for example, of the first sensor assembly 20a. Using the signal from the sensor 22, the control unit 40 determines the position of the tool relative to the landmark 32. The control unit 40 also compares the position of the tool to an acceptable range of positions, such as a range of acceptable positions of a fastener relative to the landmark 32. For example, landmark 32 can be a variable angle locking hole, and the fastener can be a bone screw configured for variable-angle locking in the variable-angle hole. The variable-angle locking screw and variable-angle locking hole may have a limited range of angles for which use is approved, or indicated for a given procedure. As another example, when the tool includes a drill bit, the control unit 40 can compare an angle of the drill bit relative to a central through axis of the variable-angle locking hole to an acceptable insertion angle of the variable-angle locking hole. Additionally, a particular medical procedure may require that a fastener be inserted at a particular angle or position relative to the landmark. For example, a surgeon or other individual may determine that a particular bone fragment is disposed at a first angle relative to a variable angle locking hole or a non-locking hole. The control unit 40 can be used to identify when the landmark identifier 10 is targeting the bone fragment such that the bone fragment can be captured and secured by a fastener.

In some implementations, the control unit 40 outputs on the graphical user interface 42 an indication that the position of the landmark identifier 10 relative to a landmark 32 is acceptable. For example, the output on the user interface 42 can include one or more elements, such as an element representing the angle of the landmark identifier 10 relative to an axis of the landmark 32, one or more elements representing acceptable positions of the landmark identifier 10 relative to the landmark 32, one or more elements representing unacceptable positions of the landmark identifier 10 relative to the landmark 32, a numeric representation of the angle of the landmark identifier 10 relative to an axis of the landmark 32, a numeric representation of the maximum acceptable insertion angle of a fastener, an element indicating that the current position of the landmark identifier 10 is acceptable, a graphical representation of an acceptable conical range of a variable angle or variable angle locking screw, and an element indicating that the current position of the landmark identifier 10 is unacceptable.

In some implementations, such as when a particularly large orthopaedic implant 30 is used, some landmarks of the orthopaedic implant 30 may be too far from the first sensor assembly 20a to be targeted using the first sensor assembly 20a. In such implementations, among others, the second sensor assembly 20b can be attached to the orthopaedic implant 30 at a location within the working volume shared by the first sensor assembly 20a for use in targeting the landmarks that are too far from the first sensor assembly 20a or outside the working volume. As shown in FIG. 5, the second sensor assembly 20b can be attached to the orthopaedic implant 30 through a small incision, which may have been made using the landmark identifier and the first sensor assembly 20a to reduce the number and size of incisions required to accomplish locking of a distal portion 30b of the orthopaedic implant 30.

The second sensor assembly 20b can then be calibrated using the location and orientation of the first sensor assembly 20a as a reference point. For example, the second sensor assembly 20b can be calibrated based on a signal received from the first sensor assembly 20a when the first sensor assembly 20a and the second sensor assembly 20b are located within the same working volume of the landmark identifier 10. The known location and orientation of the first sensor assembly 20a can be used as a reference that can be used to determine an unknown degree of freedom of the second sensor assembly 20b.

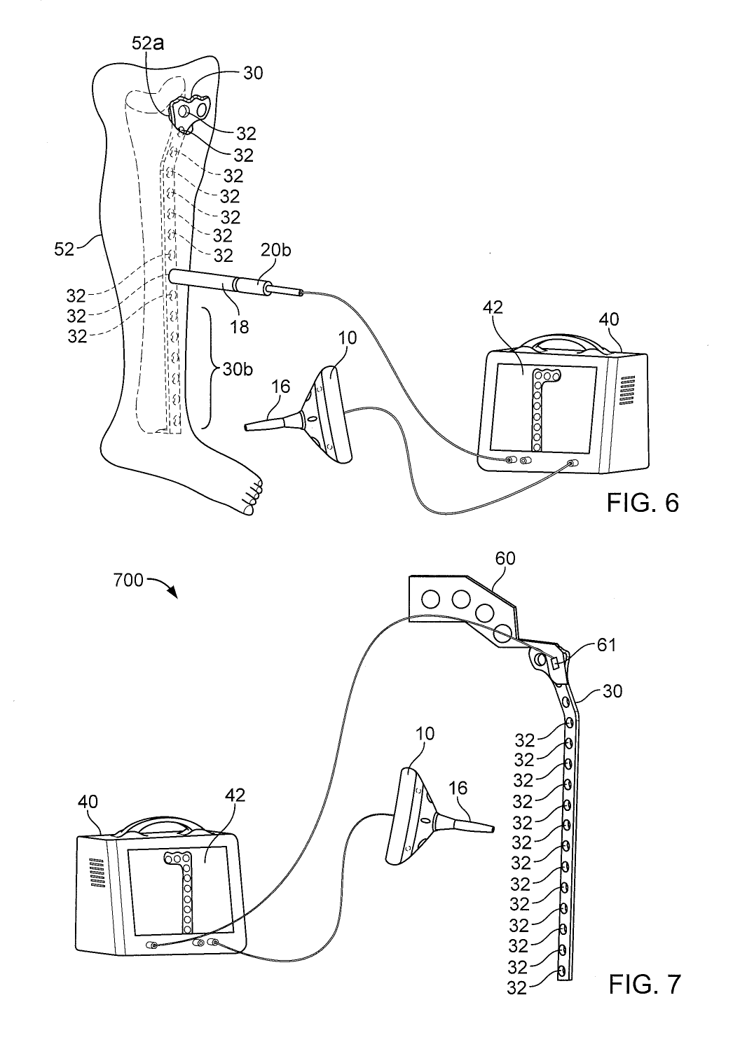

Once the second sensor assembly 20b has been calibrated, the control unit 40 can change or relocate the global reference frame of the targeting system 300 from the position of the first sensor assembly 20a to the position of the second sensor assembly 20b. This is called the leapfrog targeting technique for the sake of explanation here. For example, the control unit 40 may initially determine the position of the landmark identifier 10 with reference to the position of the first sensor assembly 20a, located at the proximal end of the bone plate 30. After the second sensor assembly 20b is attached to the orthopaedic implant 30 and calibrated, however, the control unit 40 may change its global reference frame to determine the position of the landmark identifier 10 with reference to the position of the second sensor assembly 20b instead of the first sensor assembly 20a. Landmarks 32 in the distal region 30b of the orthopaedic implant 30 can then be targeted using the second sensor assembly 20b. The first sensor assembly 20a can be removed, if desired, as shown in FIG. 6.

This relocation of the global reference frame can be repeated as many times as needed depending on the length and number of landmarks in the orthopaedic implant. An operator may choose to locate the calibrated first sensor assembly 20a in the middle of the orthopaedic implant to reduce the number of times that the operator needs to calibrate additional sensors. For example, if a plate is 95 cm long and the working volume is 50 cm wide, by placing the first sensor assembly 20a in the middle of the plate, a surgeon only needs to move the landmark identifier 10 from one side of the sensor assembly to another side without relocating the sensor assembly.

In the leapfrog technique, as the landmark identifier 10 moves relative to the second sensor assembly 20b, the signals produced by the second sensor assembly 20b change accordingly. The control unit 40 can update the user interface 42 to indicate current position of the landmark identifier 10 relative to landmarks 32 of the orthopaedic implant 30. In addition, as described above, the control unit 40 can confirm acceptable positioning of the landmark identifier 10 relative to the orthopaedic implant 30. With this information, the operator of the landmark identifier 10 can use the information to align the drill sleeve 16 and/or other tools with a landmark 32, such as a particular blind hole 32 of the orthopaedic implant 30.

FIG. 7 illustrates a system 700 for targeting landmarks. The system 700 includes a control unit 40, a landmark identifier 10, and an insertion handle 60 coupled to an orthopaedic implant 30. The insertion handle 60 can be used to maneuver the orthopaedic implant 30 during implantation in a patient. The insertion handle 60 is removably coupled to the orthopaedic implant 30, so that the insertion handle 60 can guide the orthopaedic implant 30 during implantation and then be removed from the orthopaedic implant 30 after implantation has been completed. The insertion handle 60 couples to the orthopaedic implant 30 at a fixed position relative to the bone plate 30. The insertion handle 60 also includes an electromagnetic field sensor 61 that responds to electromagnetic fields produced by the landmark identifier 10. The sensor 61 is attached to the insertion handle 60 at a known, fixed position of the insertion handle 60. Thus, when the insertion handle 60 is attached to the orthopaedic implant 30, all six degrees of freedom of the sensor 61 are known and the sensor 61 is disposed at a known location and orientation relative to the landmarks 32 of the orthopaedic implant 30.

If one of more degrees of freedom of the sensor 61 on the insertion handle 60 is not initially known, the sensor 61 of the insertion handle 60 can be calibrated using a second sensor 22 attached to the orthopaedic implant 30 with a known location and orientation relative to a landmark 32 of the orthopaedic implant 30 or relative to a known landmark of the insertion handle 60. Alternatively, the landmark identifier 10 can be attached to the orthopaedic implant 30 with a known location and orientation relative to a landmark of the orthopaedic implant 30 or relative to a known landmark of the insertion handle 60. In some implementations, the sensor 61 of the insertion handle 60 can be shipped in a pre-calibrated state such that upon attachment of the insertion handle 60 to the orthopaedic implant 30, the position of the sensor 61 relative to landmarks 32 of the orthopaedic implant 30 is known for six degrees of freedom.

In other implementations, a targeting system includes a large flat field generator disposed under the body part or the fractured bone. The targeting system also includes two sensors, one coupled to the plate and the other coupled to a drill sleeve, for example. If the generated field is larger than the volume of the largest implant intended to be used with the system, no leapfrog technique nor placement of the sensor assembly in the middle of the plate will be needed to target all of the landmarks of the plate.

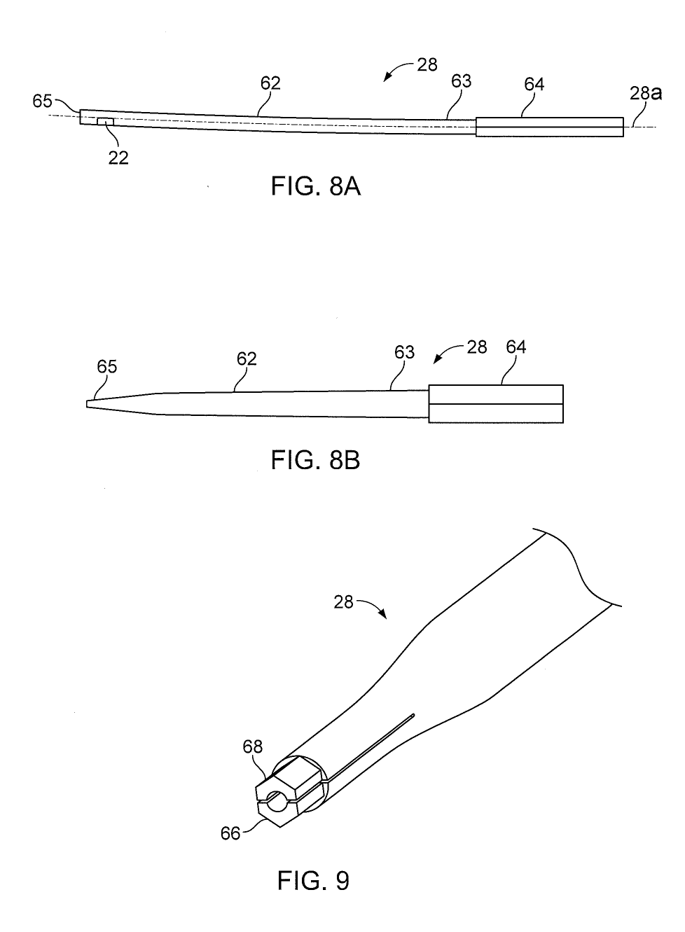

Various implementations for attaching a sensor assembly 20 to a housing 28 or attaching a housing 28 to an orthopaedic device 30 are shown in FIGS. 8A-12B. Referring to FIG. 8A, a housing 28 of a sensor assembly 20 includes an extension 62 and a head 64, and a sensor 22 located at a known position relative to the housing 28, for example, at an end 65 of the extension 62. The housing 28 is curved along a longitudinal axis 28a. For example, the extension 62 of the housing 28 can be curved such that when the extension 62 is inserted into a drill sleeve 18, the extension 62 straightens to conform to the limited space within the drill sleeve 18. The extension 62 presses against the inner surface of the drill sleeve 18 to secure the housing 28 within the drill sleeve 18 with a frictional fit. The head 64 of the housing 28 engages an end of the drill sleeve 18 to limit insertion of the housing 28 into drill sleeve 18.

Alternatively, as shown in FIG. 8B, the extension 62 can include a tapered or slightly frusto-conical outer surface. A proximal end 63 of the extension 62 located near the head 64 can feature a larger outer diameter than the distal end 65 of the extension 62. When the housing 28 is inserted into the drill sleeve 18, friction between the tapered surface of the extension 62 and the interior of the drill sleeve 18 secures the housing 28 within the drill sleeve 18. In some implementations, the drill sleeve 18 can include at tapered inner portion that provides a seat for engaging the extension 62.

As another alternative, shown in FIG. 9, the housing 28 is configured to engage a mating structure to attach the housing 28 to an orthopaedic device. The housing 28 includes a split end 66 that is expandable to engage a mating structure to fixedly attach the housing 28 to an orthopaedic device, for example, an orthopaedic implant. The split end 66 can receive a ridge or a wedge of a mating structure of an orthopaedic implant to attach the housing 28 to the orthopaedic implant. For example, the ridge or wedge of the mating structure can expand the split end 66 to create a friction fit between an exterior surface of the split end 66 and a portion of the mating structure. The housing 28 also includes a polygonal external portion 68 to mate with a complementary polygonal portion, for example, a socket, of an orthopaedic implant. In use, the polygonal external portion 68 prevents undesired rotation of the housing 28 relative to the mating structure.

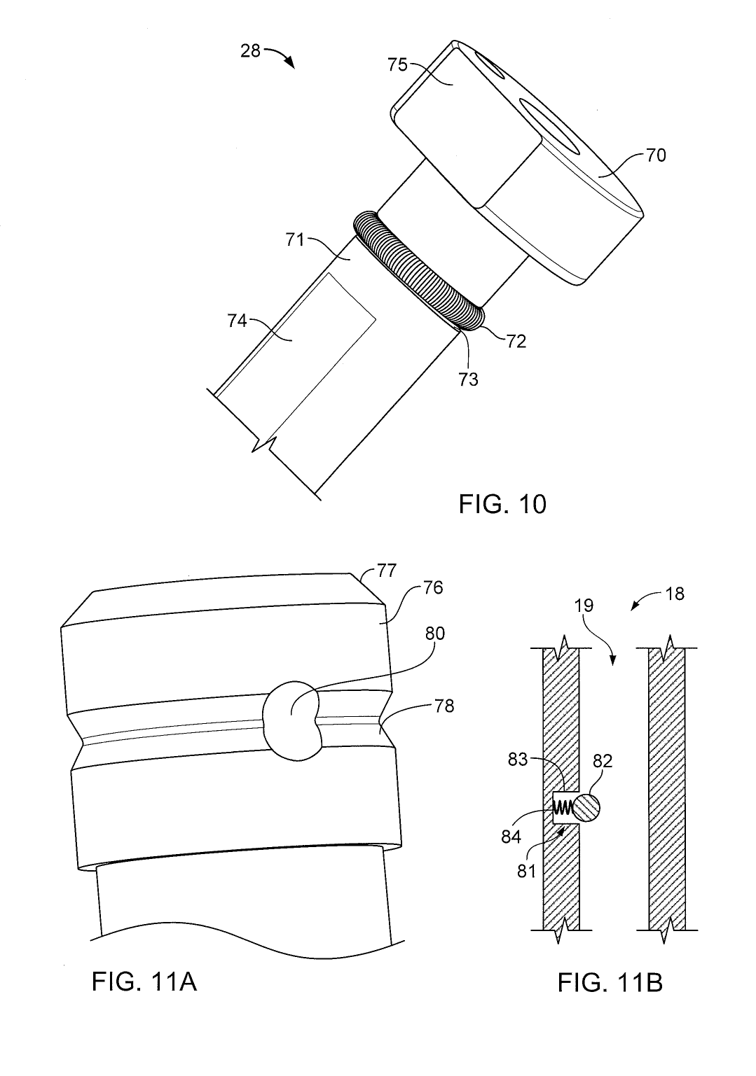

Now referring to FIG. 10, the housing 28 of a sensor assembly 20 can alternatively include a head 70 and a generally cylindrical outer surface 71. On the generally cylindrical outer surface 71, the housing 28 includes a circumferential groove 73 in which a compressible member 72, such as a spring member or an elastomeric ring, is partially disposed. When the housing 28 is inserted into a drill sleeve 18, the compressible member 72 compresses within the drill sleeve 18 and provides a frictional fit to secure the housing 28 within the drill sleeve 18. Optionally, the drill sleeve 18 can include an inner circumferential groove in which the compressible member can expand to secure the housing 28 to the drill sleeve 18. The head 70 can limit insertion of the housing 28 in the drill sleeve 18 to dispose a sensor at a known location relative to the drill sleeve 18. Additionally, the generally cylindrical outer surface 71 of the housing 28 may include a planar region 74 that can be aligned with a complimentary planar region of a drill sleeve 18 or mating structure that can limit rotation of the housing 28 within the drill sleeve 18 and/or provide a known rotational position of the housing 28 relative to the drill sleeve 18. The head 70 also includes a planar region 75 that can be aligned with a complimentary surface to provide alignment and rotational stability.

As another alternative, shown in FIGS. 11A and 11B, the housing 28 includes an end 76 configured for insertion into a drill sleeve 18. The end 76 includes an outer circumferential groove 78 and/or a spherical detent 80. The end 76 also includes a chamfered edge 77. The drill sleeve 18 (FIG. 11B) includes a ball plunger 81 located on the interior of the drill sleeve 18. The ball plunger 81 includes a ball 82 that partially extends into through hole 19 of the drill sleeve 18. The ball plunger 81 is also partially disposed within a recess 83 defined in the inner surface of the drill sleeve 18 that receives the ball 82. The ball plunger 81 includes a resilient member 84 disposed in the recess 83 that biases the ball toward the through hole and that compresses when a force is exerted on the ball 82. When the end 76 of the housing 28 is inserted into the drill sleeve 18, the chamfered edge 77 of the housing 28 depresses the ball 82 of the ball plunger 81 into the recess 83 to permit the end 76 of the housing 28 to travel over the ball plunger 81. When the circumferential groove 78 is aligned with the ball plunger 81, the ball 82 moves into the circumferential groove 78 to resist further travel of the end 76 through the drill sleeve 18. The housing 28 may be rotated until the ball 82 is received in the detent 80, which resists rotation of the housing 28 within the drill sleeve 18. Optionally, the end 76 can include the circumferential groove 78 without the detent 80, or the end 76 can include the detent 80 without the circumferential groove 78. In other implementations, the ball plunger 81 may be included on the end 76 of the housing 28 instead of on the drill sleeve 18, and an inner surface of the drill sleeve 18 may include a circumferential groove and/or a detent to receive the ball 82 Of the ball plunger 81.

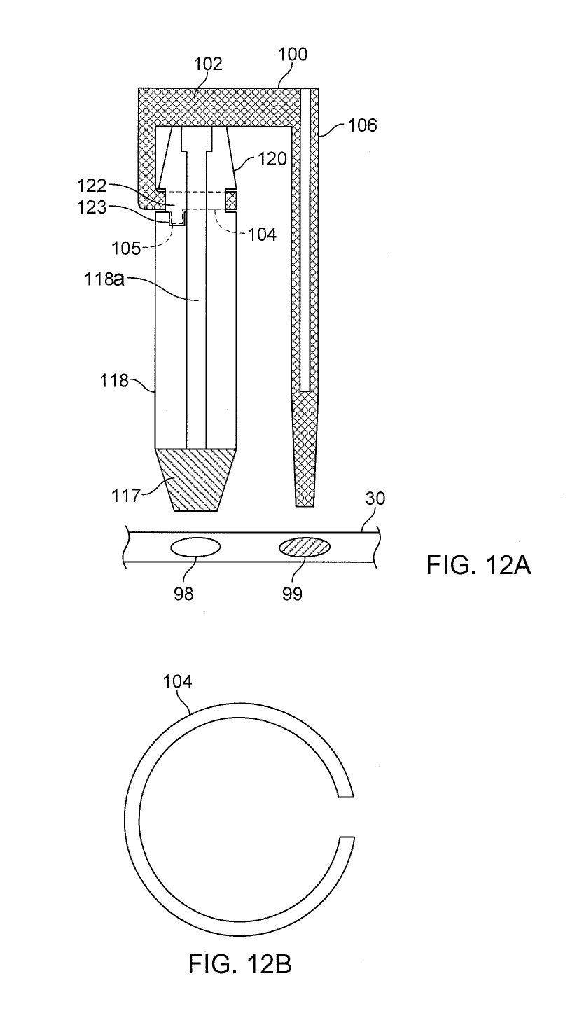

In some implementations, the housing 28 can be engaged with an orthopaedic implant by external attachment to the drill sleeve 18. For example, as shown in FIGS. 12A and 12B, an attachment member 100 can be attached to a drill sleeve 118 to provide a receptacle for the housing 28. The drill sleeve 118 includes a threaded distal end 117 that attaches to a threaded region 98 of an orthopaedic implant 30. The drill sleeve 118 includes a tapered proximal end 120 and a circumferential groove 122. The drill sleeve 118 can include a slot 123 located in or near the groove 122 on the exterior of the drill sleeve 118. The drill sleeve 118 also includes a through hole 118a to admit tools and/or fasteners, such as bone pins.

The attachment member 100 includes an arm 102 connecting a split ring 104 (FIG. 12B) and a tubular portion 106. The split ring 104 is configured to engage the groove 122 of the drill sleeve 118. The attachment member 100 can also include a tab 105 located on or near the split ring 104. To couple the attachment member 100 to the drill sleeve 118, the split ring 104 is placed over the tapered end 120 of the drill sleeve 118. Because the split ring has an inner diameter smaller than the outer diameter of the drill sleeve, the split ring 104 flexes outward as the attachment member 100 travels along the tapered end 120. The split ring 104 continues to travel until the split ring 104 is received into the groove 122 to secure the attachment member 100 to the drill sleeve 118. The tab 105 of the attachment member 100 engages the slot 123 of the drill sleeve 118 to limit rotation of the attachment member 100 relative to the drill sleeve 118. Alternatively, a tab (not shown) may be formed on the exterior of the drill sleeve 118, for example, in the groove 122, to engage the break in the split ring 104 to limit rotation of the attachment member 100 relative to the drill sleeve 118.

The tubular portion 106 of the attachment member 110 receives the housing 28 of a sensor assembly 20 or includes a sensor 22. Thus, attachment of the attachment member 110 to the drill sleeve 118 secures the sensor 22 to the drill sleeve 118 at a known position relative to the drill sleeve 118 and the bone plate 30. The attachment member 100 can be a component of a housing 28 or of a sensor assembly 20. Alternatively, the arm 102 and the tubular portion 106 can be integral with the drill sleeve 118.

The arm 102 of the attachment member 100 can be formed so that the attachment member does not block access to the through hole 118a of the drill sleeve 118 when the attachment member 100 is coupled to the drill sleeve 118. As a result, using the attachment member 100, a sensor assembly 20 can be coupled to the drill sleeve 118 without preventing access to the interior of the drill sleeve 118. In addition, the attachment member 100 may be formed so that the tubular portion 106 enters or engages a landmark 99 of the bone plate 30 when the attachment member 100 is attached to the drill sleeve 118 and the drill sleeve 118 is attached to the bone plate 30. Alternatively, the arm 102 can be configured to engage the through hole 118a to ensure correct alignment of the sensor relative to the drill sleeve 118 upon attachment and to limit rotation between the sensor 22 and the drill sleeve 118.

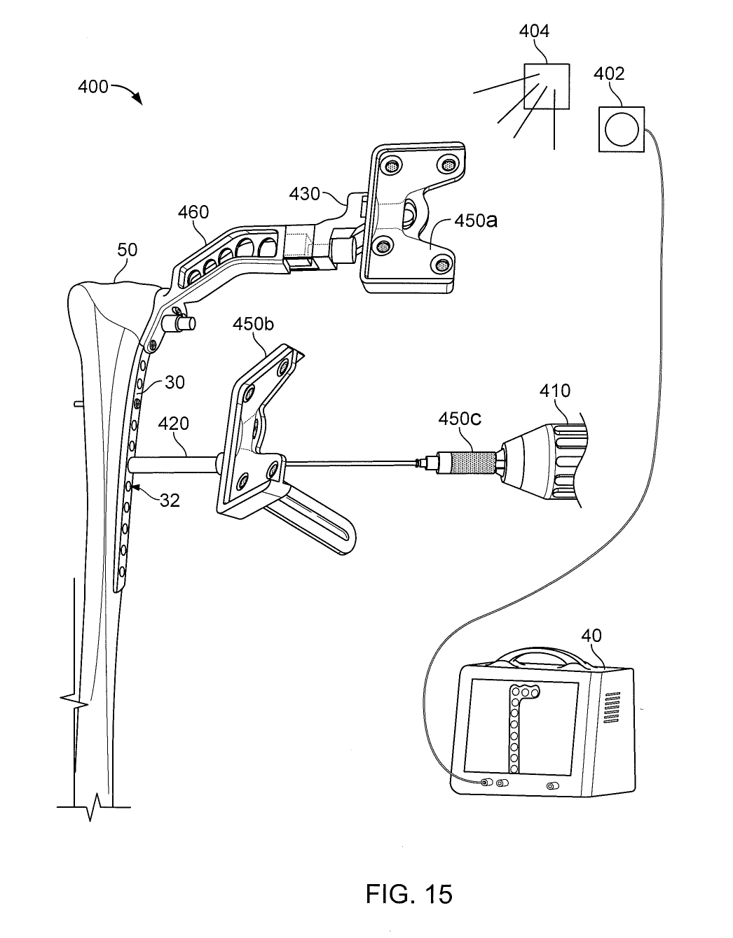

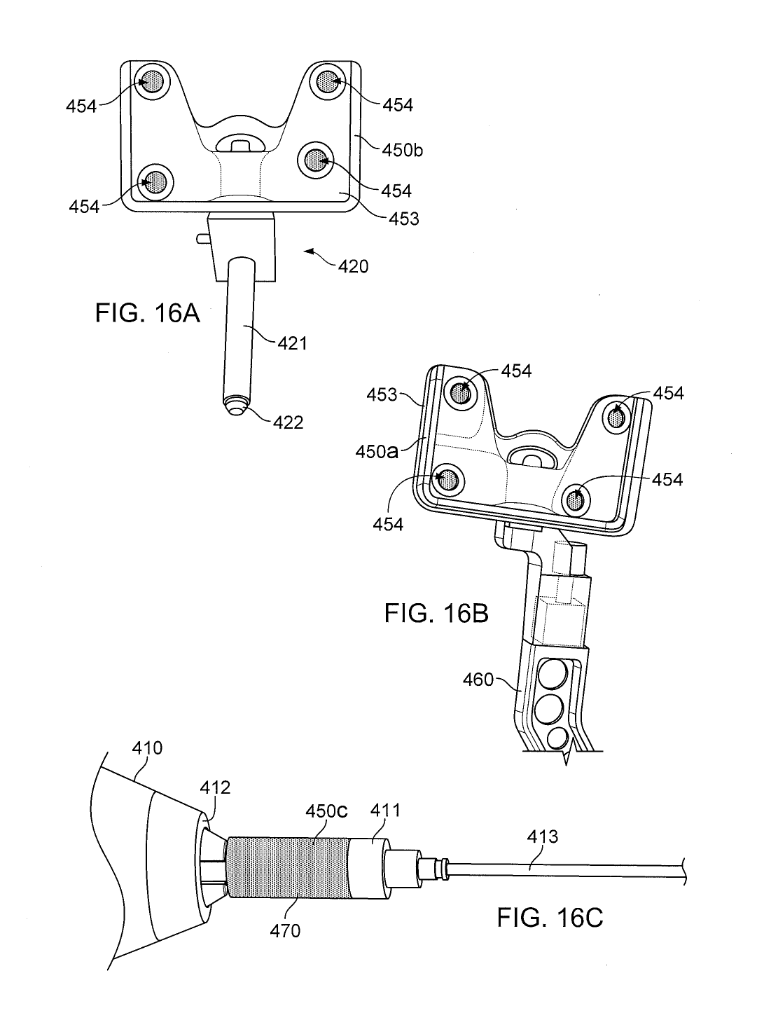

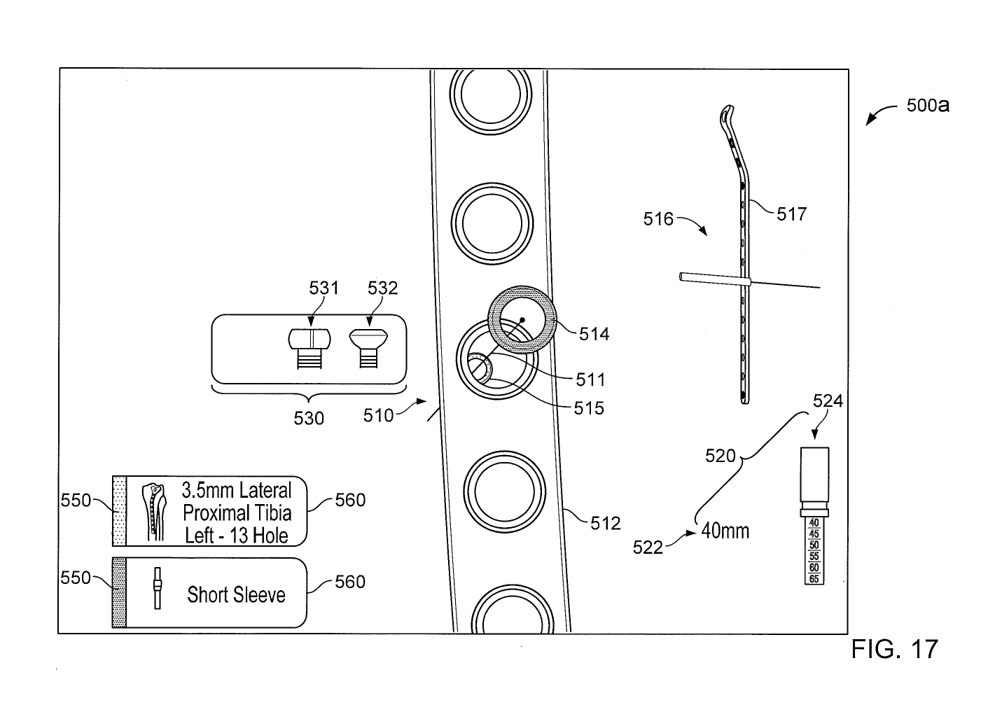

Referring to FIGS. 15 and 16A to 16C, a system 400 uses optical tracking to locate screw holes and determine the trajectory of drilling or screw insertion during surgery. The system 400 includes an infrared (IR) camera 402 in communication with the control unit 40 and an IR light source 404. The camera 402 detects IR light that is reflected from fiducials in the field of surgery. The control unit 40 receives signals from the camera 402 that indicate the reflected light from the fiducials. Using the signals from the camera 402, the control unit 40 determines the relative positions of the fiducials. The fiducials are attached to various instruments at known, fixed positions, such that the positions of the instruments can be determined from the positions of the fiducials.

The system 400 includes a drill 410 or other instrument and a guide 420 and a handle 460 for coupling to an orthopaedic implant 30. A fiducial 450a-450c is coupled to each of the drill 410, the guide 420, and the handle 460. The fiducials 450a-450c can be coupled directly to an instrument, or can be coupled to an instrument through another component. For example, as illustrated, the fiducial 450a attaches to the end of an insertion handle 460 that can be used to implant the orthopaedic device 30. As an alternative, the fiducial 450a can be configured to attach directly to the orthopaedic device 30.

In some implementations, the fiducials 450a-450c are removable components that can be attached and detached from the insertion handle 460, the guide 420, and the drill 410, respectively. In some implementations, the fiducials 450a-450c are formed as fixed components that are formed to be integral with the insertion handle 460, the guide 420, and the drill 410, respectively.

Referring to FIGS. 16A and 16B, each of the fiducials 450a, 450b includes a housing 453 and a reflective material, such as a foil, located within the housing 453. The housing 453 defines openings 454 that expose the reflective material. In some implementations, the fiducials 450a, 450b define three, four or more openings. Each fiducial 450a, 450b can include a different spacing between or configuration of the openings 454, permitting the control unit 40 to distinguish between the fiducials 450a, 450b and thus distinguish the instruments to which the fiducials 450a, 450b are attached, as well as allow the camera to track the location and orientation of the fiducials 450a, 450b.

In some implementations, rather than including openings along a plane, the fiducials 450a-450c can include an arrangement of a plurality of reflective spheres or other elements. In some implementations, the fiducials 450a, 450b emit IR light. The fiducials 450a, 450b can be an integral part or a separate part of the components to be tracked.

The guide 420 includes a sleeve 421 that defines an internal channel that receives a drill bit or other instrument. The sleeve 421 can guide a drill bit and can protect tissue surrounding an operation site. A tip 422 of the sleeve 421 is dimensioned to engage a landmark of the orthopaedic implant 30. For example, the tip 422 is sized to enter one of the holes 32 of the orthopaedic implant 30. In particular, the tip 422 is dimensioned to remain in or contact the hole 32 while an operator adjusts the angle of the guide 420 relative to the implant 30 or relative to the axis of the hole 32. With the tip 422 positioned in one of the holes 32, an operator can tilt the relative to the implant 30 to achieve a desired angle for, for example, drilling or screw insertion. Thus the location at the hole 32 can be maintained while the orientation of the sleeve 421 is adjusted relative to the hole 32.

The fiducial 450a attaches to the end of the insertion handle 460. Because the fiducial 450a is coupled to the insertion handle 460 at a known position, the control unit 40 can determine the position of the orthopaedic implant 30 based on the position of the fiducial 450a. An operator can attach the fiducial 450a to the insertion handle 460 after the orthopaedic implant 30 is implanted, and while the insertion handle 460 is still attached to the orthopaedic implant 30.

Referring to FIG. 16C, the fiducial 450c includes a band of reflective material wrapped about a drill connection 411 or a drill bit 413. As shown, a reflective band 470 is located about a drill connection 411 that is coupled between the chuck 412 of the drill 410 and a drill bit 413. The fiducial 450c can define a position along the axis of the sleeve 421 of the guide 420, and the axis of the sleeve 421 can be determined based on the position of the fiducial 450b.

The fiducial 450c can alternatively be located on the chuck 412, the drill bit 413, or other portions of the drill 410 at a known location. As another alternative, other types of fiducials can be used with the drill 410, for example, a fiducial that includes reflective material in a plane, or an arrangement of reflective elements.

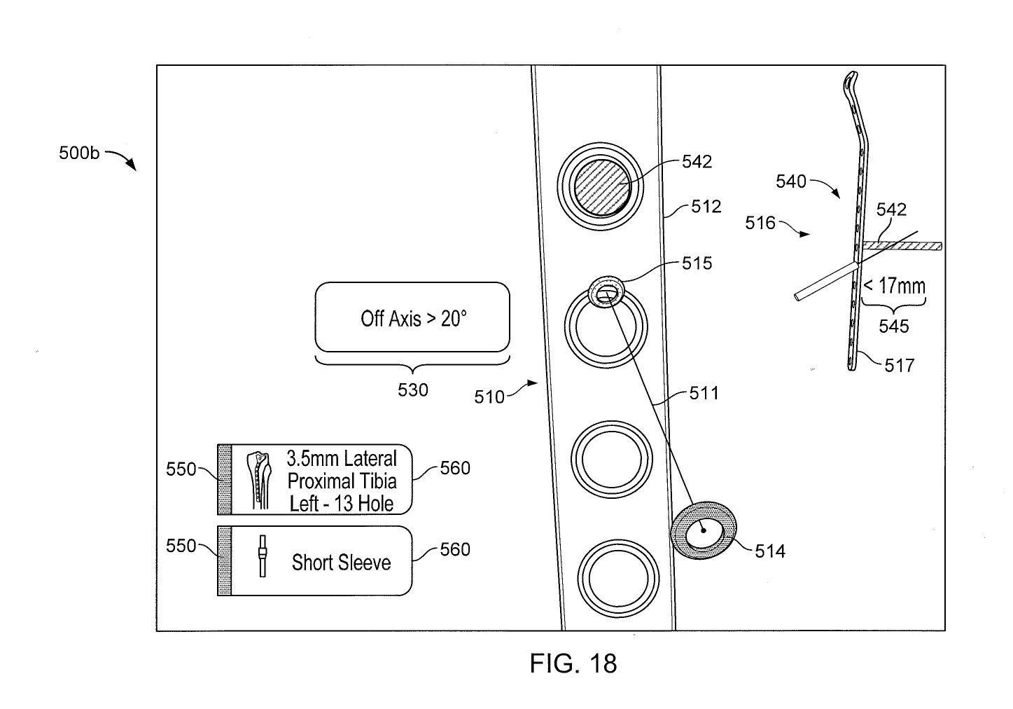

Referring to FIGS. 17 and 18, examples of user interfaces 500a, 500b for the control unit 40 are shown. As an operator positions the drill 410 and the guide 420, the control unit 40 determines the positions of the drill 410 and the guide 420 relative to the orthopaedic implant 30 or relative to the axis of the hole 32. The control unit 40 displays information about the relative positions on, for example, the user interface 500a or the user interface 500b. The control unit can track locations and orientations of the components attached to the fiducials 450a-450c. As the relative positions change, the control unit 40 detects the change using the signals from the camera 402, calculates the current positions, and displays updated information on the user interface 500a or the user interface 500b.

The features described with respect to the user interface 500a of FIG. 17 can also be included in the user interface 500b of FIG. 18, and vice versa. The user interface 500a illustrates output of the control unit 40 for the system 400 before a hole is drilled or a screw is inserted. The user interface 500b illustrates output of the control unit 40 with the elements of the system in different relative positions and after a hole has been drilled or a screw has been inserted using the system 400.

To permit calculation of the relative positions of the drill 410, the guide 420, and the orthopaedic implant 30 the fiducials 450a-450c should remain in view or line of sight of the camera 402. If one of the fiducials becomes obstructed, for example, and is no longer within view of the camera 402, the control unit 40 can indicate the obstruction to the user.

The control unit 40 can indicate the relative position of the guide 420 or other instrument relative and the orthopaedic implant 30. The surgeon can use the relative position to locate screw holes 32 or other landmarks. After using the system 400 to located a hole 32, the operator can create an incision over the hole 32 and can position the guide 420 such that the tip 422 engages the hole 32. The operator can then use the system 400 to position the guide 420 relative to the hole 32 for drilling and for insertion of a screw or other transfixion component.

The control unit 40 can provide a variety of indicators on a single user interface 500a or user interface 500b. The user interfaces 500a, 500b can each include one or more of, for example, a trajectory indicator 510, a screw length indicator or drill depth indicator 520, a component or screw type selection indicator 530, a component or trajectory collision indicator 540, a status indicator 550, and a configuration indicator 560. Any combination or subcombination of the indicators can be simultaneously displayed on a screen or other display, or across multiple displays.

The trajectory indicator 510 indicates the current trajectory defined by the guide 420. The trajectory indicator 510 can include a trajectory line 511 that represents the orientation of the sleeve 421 relative to the orthopaedic implant 30 or relative to the axis of the hole 32, indicating the trajectory currently defined by the guide 420. A representation 512 of the orthopaedic implant 30 can be displayed, and the trajectory line 511 can be displayed relative to the representation 512 in three dimensions.

The trajectory indicator 510 can also include an element 514, such as a circle, that represents the position of the proximal end of the sleeve 421. The trajectory indicator 510 can include an element 515 that represents the position of the distal end or tip 422 of the sleeve 421, which can be displayed as a circle that is smaller than the element 514.

The trajectory line 512 can be defined between the elements 514, 515. In some implementations, to align the guide 420 at a desired axis relative to the orthopaedic implant 30 or the axis of the hole 32, the operator moves the guide 420 such that the elements 514, 515 coincide. This position can represent, for example, an alignment perpendicular to the orthopaedic implant 30 or coaxial with the axis of the hole 32.

Multiple trajectory indicators can be simultaneously displayed. For example, the trajectory indicator 510 can indicate a drilling trajectory with respect to a representation 512 that is zoomed in to show a front view of a portion of the orthopaedic implant 30 near the tip 422 of the guide 420. A second trajectory indicator 516 can display, for example, a representation 517 of the entire orthopaedic implant 30. The second trajectory indicator 516 can thus provide context to the operator, indicating the trajectory and the position of a landmark of interest relative to the orthopaedic implant 30 as a whole. The second trajectory indicator 516 can also display a second view of the orthopaedic implant 30, for example, a side view. Either or both of the trajectory indicators 510, 516 can be displayed on the user interface 500a or the user interface 500b.

The drill depth indicator 520 indicates the depth that a drill bit has been inserted into tissue contacting the orthopaedic implant 30. For example, the drill depth indicator 520 can indicate the distance that the drill bit has passed into the bone 50. As the operator drills along a trajectory, a numerical indicator 522 indicates the current drill depth. A graphical indicator 524 also indicates the drill depth, for example, by displaying a representation of markings or graduations that are shown on instruments.

The control unit 40 can calculate the current drill depth by determining the relative position of the fiducial 450c on the drill 410 and the fiducial 450b on the guide 420. The control unit 40 can store information that indicates, for example, the distance between the fiducial 450c and the end of the drill bit 413, and also the distance between the fiducial 450b and the tip 422 of the sleeve 421. When positioned at the hole 32, the tip 422 can have a known position relative to the bone. For example, the tip 422 can be located at the surface of the bone 50. As the operator moves the drill bit 413 through the sleeve 421, the control unit 40 calculates the relative positions of the fiducials 450b, 450c. Using these relative positions, the control unit 40 calculates the drill depth as the distance that the end of the drill bit 413 extends beyond the tip 422 of the sleeve 421. The drill depth can be used for screw length selection. For example, the operator can select a screw that has a length substantially equal to a drilled depth. Thus the drill depth indicator 520 can be used to indicate a screw length for a screw to be inserted.

The component selection indicator 530 indicates components or component types, such as locking vs. non-locking screws, or polyaxial vs. monoaxial screws, that can be used with the current trajectory defined by the guide 420 relative to the orthopaedic implant 30. As illustrated, graphical representations 531, 532 can be displayed to represent components or techniques that can be used with the trajectory of the guide 420 as currently defined. Text or other symbols can also indicate the components that can validly be used at the current trajectory.

Various transfixion components are only indicated for use at particular trajectories or angle ranges. For example, a monoaxial screw may only be indicated for insertion perpendicular to the orthopaedic implant 30. In the illustrated example, the current trajectory is not perpendicular to the orthopaedic implant. As a result, a representation of a monoaxial locking screw, for example, a screw that includes threads on the screw head, is omitted from display in the component selection indicator 530. The omission can indicate, for example, that the monoaxial locking screw is not indicated for use, or that a likelihood of success using the monoaxial locking screw at the current trajectory is below a minimum threshold.

By contrast, the component selection indicator 530 displays a representation 531 of a polyaxial (e.g., variable angle) screw with a deformable head and a representation 532 of a polyaxial non-locking screw to indicate that either of these polyaxial screws may be selected for use at the current trajectory. As the operator adjusts the trajectory for drilling, the control unit 40 calculates the components or types of components that can be used for the current trajectory, and updates the component selection indicator 530 to indicate the current range of components that can be used. As a result, as the operator tilts the guide 420 through multiple trajectories, representations 531, 532 of components may appear or disappear to indicate the valid component options that can be selected for the trajectories. As shown in FIG. 18, the component selection indicator 530 can also indicate whether the guide 420 is positioned such that no valid components can be used at the current trajectory.

The user interface 500b of FIG. 18 includes a trajectory or screw collision indicator 540 that indicates when an orientation of the guide 420 result in a trajectory that would interfere with a placed screw, a previously drilled hole, or other path. When a hole is drilled or a transfixion element is placed using the system 400, the control unit 40 stores the position and depth of the event. For example, the control unit 40 can store the position of a previously drilled hole relative to the orthopaedic implant 30. The control unit 40 can indicate the position of the drilled hole or screw as a visual element 542 relative to one or both of the representations 512, 516 of the orthopaedic implant 30.

As the orientation of the guide 420 changes, the control unit 40 determines whether the current trajectory intersects an obstacle, such as a previous drilled hole or an implanted screw. If the current trajectory is determined to interfere with an obstacle, for example, by intersecting a shaft of a placed screw, the collision indicator 540 displays a warning. The warning can be, for example, a color change or visual element on the user interface 500a or the user interface 500b.

When the control unit 40 determines that the guide 420 is positioned in a manner that it may interfere with an obstacle, the control unit 40 calculates the greatest distance that can be drilled along the current trajectory without causing interference. The control unit 40 displays a length indicator 545 that indicates, for example, a constraint on the length of an element that is inserted along the current trajectory. In some implementations, the length indicator 545 indicates the longest length of an element that can be used without causing interference or meeting an obstruction, for example, the longest screw that falls short of the obstruction.

The user interfaces 500a, 500b can also display one or more status indicators 550 that indicate whether elements of the system 400 are currently being tracked by the control unit 40. The status indicators 550 can be, for example, colored bars near a representation of a component of the system 400. Each status indicator 550 can be associated with a particular element of the system 400. For example, one status indicator 550 can be associated with the orthopaedic implant 30 and the fiducial 450a, and another status indicator 550 can be associated with the guide 420. When the fiducials 450a-450c are in view of the camera 402, for example, the status indicators 550 indicate that proper spatial tracking is in progress, for example, with the color green. When the control unit 40 determines that one of the components is not being accurately tracked, for example, when a fiducial 450a-450c is obstructed, the associated status indicator 550 indicate the disruption in tracking, for example, by changing to the color red. By changing color or through other representations, the status indicators 550 indicate to the operator which components of the system 400 may need to be adjusted to restore accurate spatial tracking.

The control unit 40 can also display one or more configuration indicators 560 on the user interfaces 500a, 500b. The configuration indicators 560 indicate the current configuration of the system 400, for example, indicating the particular orthopaedic implant 30 and guide 420 that are being used. The operator can select the orthopaedic implant 30, the guide 420, the insertion handle 460, and other components using an on-screen interface. The control unit 40 can store a library of components and instruments from which the operator can select the components to be used. Dimensions and properties of the various components and instruments can be stored by the control unit 40 to calculate positions between position of different combinations of instruments and components. The operator may also press the configuration indicators 560 to make a new selection during a procedure, for example, to indicate a change from using the guide 420 to a guide with different dimensions.

As an alternative to using optical tracking, the system 400 may alternatively use tracking by electromagnetic field sensors. Electromagnetic field sensors can be used in place of the fiducials 450a-450c. When located within a working volume of electromagnetic fields produced by an electromagnetic field generator, the control unit 40 can use signals from the sensors to determine relative positions, and can display the information shown on the user interfaces 500a, 500b.

In some implementations, the fiducials 450a-450c can be removable and disposable. In some implementations, the fiducials 450a-450c are autoclavable and reusable. The fiducial 450a can be preassembled as part of the handle 460 during manufacturing, and the fiducial 450b can be preassembled to the guide 420. In some implementations, the camera 402 can communicate wirelessly with the control unit 40.

A number of implementations and alternatives have been described. Nevertheless, it will be understood that various modifications may be made without departing from the spirit and scope of the disclosure. Accordingly, other implementations are within the scope of the following claims.

What is claimed is:

1. A device for targeting a landmark of an orthopaedic implant, the device comprising:
a housing configured to engage a mating structure for attachment of the housing to the orthopaedic implant; and
an electromagnetic sensor located at a known position within the housing, wherein, when the housing is engaged with the mating structure, the position of the sensor relative to a landmark of the orthopaedic implant is known for at least five degrees of freedom.

2. The device of claim 1, wherein the housing includes one of a generally cylindrical outer surface having a detent and a ball plunger to fixedly attach the housing to the implant.

3. The device of claim 1, wherein the housing includes a split end that is expandable to engage the mating structure and fixedly attach the housing to the implant.

4. The device of claims 1, wherein the housing includes an outer surface having a compressible member configured to engage a groove to fixedly attach the housing to the implant.

5. The device of claims 1, wherein the housing includes a tapered outer surface configured to engage a seat to fixedly attach the housing to the implant.

6. The device of claims 1, wherein the housing defines a central longitudinal axis, the housing having a curvature along the central longitudinal axis of the housing.

7. The device of claims 1, wherein the mating structure includes a polygonal external portion and wherein the housing includes a complimentary polygonal portion for mating with the polygonal external portion.

8. A method of targeting a landmark of an orthopaedic device, the method comprising:
locating a first landmark of the orthopaedic device using a landmark identifier and a first electromagnetic field sensor, the landmark identifier having an electromagnetic field generator, and the first electromagnetic field sensor being within a working volume of the electromagnetic field generator when locating the first landmark;
placing a second electromagnetic field sensor in the working volume; and
locating a second landmark of the orthopaedic device using the landmark identifier and the second electromagnetic field sensor.

9. The method of claim 8, wherein the first electromagnetic field sensor is located outside the working volume of the electromagnetic field generator when locating the second landmark.

10. The method of claim 8, wherein the orthopaedic device is a bone plate.

11. The method of claims 8, wherein the first landmark is a hole.

12. The method of claim 10, wherein attaching the second electromagnetic field sensor includes accessing the hole and attaching the second electromagnetic field sensor to the orthopaedic device via the hole.

13. The method of claim 11, wherein the hole is accessed when the orthopaedic device is implanted in a patient.

14. The method of claims 10, wherein the hole is a threaded hole, and wherein attaching the second electromagnetic field sensor includes engaging a drill sleeve with the threaded hole and attaching the second electromagnetic field sensor to the drill sleeve.

15. A method of confirming acceptable positioning of a tool relative to an orthopaedic stabilization structure, the method comprising:
receiving a signal from a sensor, the signal being indicative of a position of the tool relative to a landmark of the orthopaedic stabilization structure;
determining the position of the tool relative to the landmark;
comparing the position of the tool to an acceptable range of positions of a fastener relative to the landmark;
determining that the position of the tool relative to the landmark corresponds to an acceptable position within the range of positions of the fastener relative to the landmark; and
outputting on a graphical user interface an indication that the position of the tool relative to the landmark is acceptable.

16. The device of claim 1, wherein the housing further comprises two portions configured to engage two separate holes.

17. The device of claim 1, wherein the housing comprises a drill sleeve.

18. The device of claim 17, further comprises an extension.

19. The device of claim 1, further comprising a control unit, and wherein the control unit indicates an angular position of the housing relative to the orthopaedic implant.

20. The device of claim 19, wherein the control unit compares the angular position with an acceptable rang of positions.
